(19) United States
(12) Plant Patent Application Publication
(10) Pub. No.: US 2010/0251440 P1
(43) Pub. Date: Sep. 30, 2010

(54) Apple Tree Named 'WA 2'

(50) ;

(75) Inventor: Bruce H. Barritt, Okanagan Centre, CA (US)

Correspondence Address: MORRISON & FOERSTER LLP, 425 MARKET STREET, SAN FRANCISCO, CA, 94105-2482, US

(73) Assignee: Washington State University, Pullman, WA (US)

(21) Appl. No.: 12/383,556

(22) Filed: Mar. 24, 2009

(51) Int. Cl. A01H 5/00

(52) U.S. Cl. PLT/162

(57) ABSTRACT

A new and distinctive variety of an Malus domestica ‘apple’ tree, named ‘WA 2’, that is distinguished by its attractive blush, outstanding texture being firm, crisp and juicy, and the presence of the homozygous genes for ethylene production ACSI-2/2 and ACO1-2/2 which contribute to maintaining the fruit quality over long periods of storage.

### LATIN NAME OF THE GENUS AND SPECIES OF THE CLAIMED PLANT

Botanical/commercial classification: Malus domestica/apple tree

Varietals denomination: ‘WA 2’ seedling designation T19-17-3-9427

The invention refers to a new plant variety of apple tree (Malus domestica) named ‘WA 2’. This new variety is distinguished by the distinctive blush of its fruit, with the texture being firm, crisp and juicy even after 60 days of regular cold storage due to the presence of the homozygous genes for ethylene production, ACS1-2/2 and ACO1-2/2.

### BACKGROUND OF THE INVENTION

‘WA 2’ (seedling designation T19-17-3-9427) originated from a seed collected in 1994 from fruit of the ‘Splendour’ cultivar. The male parent is unknown although the ‘Tenroy’ cultivar of ‘Gala’ apple trees surrounded the ‘Splendour’ tree and may have been the pollen source. The germinated seedling was grown in a greenhouse at the Washington State University Tree Fruit Research and Extension Center (WSU TFREC) in Wenatchee, Wash. during the Summer of 1995. In September 1995, a bud from the seedling was budded to M9 rootstock and the resulting ‘mother’ tree was planted in the evaluation orchard at WSU TFREC in the Spring of 1997. Fruit from the ‘mother’ tree were evaluated in 2000 and 2001. Second generation trees were produced by chip-budding to M9 rootstock Fall of 2002 and the resulting trees planted at three locations in Washington State in 2004 near Chelan, Wash., Chelan County; near Wenatchee, Wash., Douglas County; and near Basin City, Wash., Franklin County. Fruit from the second generation trees at all three orchard locations were compared to that of the ‘mother’ tree in 2005, 2006, 2007, and 2008, and found to be essentially similar to that of the ‘mother’ tree.

### SUMMARY OF THE INVENTION

‘WA 2’ is an attractive apple that is distinct in appearance from that of other commercially grown apples and has outstanding texture, being very firm, crisp and juicy, both fresh and after 60 days of regular cold storage. Firmness is comparable to the commercial varieties of ‘Cripps Pink’ and ‘Braeburn’ and is firmer than the commercial varieties ‘Gala’ and ‘Fuji’ (Table 1, second column). Fruit of ‘WA 2’ is sweet with percent soluble solids greater than for ‘Gala’ and ‘Braeburn’ and is comparable to ‘Fuji’ and ‘Cripps Pink’ (Table 1, third column). Fruit acidity level of ‘WA 2’ is intermediate between low acidity cultivars ‘Gala’ and ‘Fuji’ and high acidity cultivars ‘Braeburn’ and ‘Cripps Pink’ (Table 1, fourth column). Overall flavor is well balanced between sugar and acid. ‘WA 2’, like its maternal parent, is homozygous for the ethylene production gene Md-ACS1. In addition it is homozygous for a second ethylene production gene Md-ACO1. The ACS and ACO genotypes were determined by fragment analysis of the results of polymerase chain reactions using the primers published by Harada et al. (2000) and Costa et al. (2005).

### BRIEF DESCRIPTION OF THE DRAWINGS

FIG. 1. The eight year old mother tree of ‘WA 2’ on M9 rootstock near full bloom.

FIG. 2. ‘WA 2’ mother tree fruit at harvest maturity.

FIG. 3. View of ‘WA 2’ fruit showing exposed and backsides, and stem and calyx ends.

FIG. 4. View of typical ‘Tenroy Gala’, ‘WA 2’ and ‘Splendour’ apples at harvest maturity.

### DETAILED BOTANICAL DESCRIPTION

The following detailed description is from the ‘mother’ tree grown at WSU TFREC. All color references are from the R.H.S. colour chart by The Royal Horticultural Society.
Tree:
Type: Considered spur type with development mostly on 2 yr. old wood, some on current seasons growth; spur length ranges from 1 to 8 cm
   Vigor.—considered moderate.
   Overall shape.—upright and spreading.
   Height.—14 feet.
   Width.—7 feet.
   Hardiness.—considered hardy for the regions grown in.
   Productivity.—considered very productive, exhibits alternate bearing tendencies if over-cropped the previous season.
Trunk:
   Size.—diameter at a height of 45 cm is 11.4 cm.
   Bark texture.—smooth.
   Bark color.—from the greyed-orange group (177A).
   Lenticels.—numerous and horizontal to the plane, size averages 9.9 mm×1.8 mm.
   Lenticels color.—from the brown group (200C).
Branches:
   First year branches.—considered moderate in number with light spur development; spreading habit with moderate crotch angles of 0° to 35° Color: from the greyed-purple group (187A) Lenticels: round to slightly elongated and oriented parallel to growth with color from the white group (N155D); size average is 0.5 mm×1 mm; density is at 11 per running cm Branch pubescence: present, moderate, with color from the white group (155D) Internodes: spacing averages 4.2 cm.
   Scaffold branches.—moderate in number; spreading habit with crotch angles of 30°-80° Color: from the grey-brown group (N199D) Lenticels: numerous and horizontal to plane, rough; averages 10.2 mm×2.1 mm in size.
   Lenticels color.—from the white group (N155C).
Leaves:
   Shape.—considered oval.
   Texture.—considered leathery.
   Sheen.—upper surface is glossy.
   Pubescence.—lower surface is covered with fine pubescence.
   Length.—ranges from 7.2 to 10.5 cm; average length is 9.2 cm.
   Width.—ranges from 4.6 to 7.3 cm; average length is 6.0 cm.
   Margin.—considered double serrate.
   Tip.—considered cuspidate.
   Base.—considered obtuse.
   Stipules.—present on most leaves; average number per leaf is 0.8 with a range of 0 to 2 per leaf; average length is 6.3 mm; width ranges from 0.8 to 1.0 mm.
   Leaf color.—upper surface from the yellow-green group (147A); lower surface from the yellow-green group (147B).
   Petiole.—lengths range from 2.2 to 3.9 cm, average is 3.3 cm; average diameter is 1.7 mm; color is from the green group (142D) with high lights from the greyed-purple group (186A).
Flowers: Full bloom date May 5, 2008 at Wenatchee, Wash.; number of blossoms per bud ranges from 4 to 6 — mostly 5 — with an average of 4.6 blossoms per bud
   Size.—individual flowers considered medium in size averaging 50.2 mm across.
   Petal.—width averages 17.9 mm; length averages 21.1 mm.
   Color.—from the white group (N155B) with highlights from the greyed-purple group (186D); petal vein color from the greyed-purple group (186B).
   Stamen.—range of 20-22 mm length with an average length of 20.7 mm Filament: color is from the green-yellow group (1D) Anthers: color at maturity from the yellow group 4C; average length is 2.1 mm.
   Pistil.—average length is 7.9 mm Styles: 4 to 6 in number generally 5, fused at base and pubescent at union; average length is 3.2 mm; color is from the yellow-green group (154C) Stigma: club shaped; color is from the yellow-green group (154B).
   Sepals.—five per bloom and occasionally curled back towards the peduncle; average length is 7.2 mm and average width is 4.2 mm; colorless pubescence is present on both upper and lower surfaces; sepal color is from the green group (138B) with tips highlighted from the greyed-red group (178B).
Fruit: Observations and testing from mother tree fruit
   Size.—considered medium averaging 7.7 cm in equatorial diameter and 7.0 cm in axis diameter.
   Form.—considered round-conical.
   Stem.—medium reaching above stem bowl and averaging 2.3 cm in length; medium stout averaging 3.3 mm in diameter at midpoint; exhibits pronounced clubbed end.
   Stem cavity.—average width is 2.9 cm; average depth is 1.7 cm.
   Cavity shape.—acuminate.
   Lipped.—no.
   Basin cavity.—average width is 31.2 mm; average depth is 17.6 mm; some light ribbing.
   Calyx.—eye is generally erect.
   Skin.—smooth with bloom present.
   Appearance.—blush over 75-95% of surface.
   Lenticels.—numerous, distinct, ranging from 6-15 per square cm.
   Lenticels size.—round and ranging from 0.3-0.7 mm in diameter; color from the white group (157D).
   Skin color.—blush color is from the red group (47A); undercolor is from the yellow group (4C).
   Core.—core line attachment is medium.
   Core position.—is distant.
   Cell.—Tufted: no Cell Shape: obovate.
   Tube.—funnel shaped.
   Sepals.—downy.
   Stamen position.—medium.
   Axis.—axile and closed.
   Seed.—Number: 1-4 per cell with an average of 3 Shape: acute; size averages 4.5 mm in diameter×8.1 mm in length Color: from the brown group (200D).
   Flesh.—firm, crisp, melting texture, considered mildly acidic with excellent sugar balance; color from the yellow-white group (158D).
   Aroma.—mild, apple like.
   Date of harvest maturity.—Aug. 6, 2008; five year average is August 2.
   Maturity indices.—Starch: Cornel chart is 3.6 Pressure: in Newtons is 82.8 Soluble Solids: in °Brix is 12.3 Titratable Acid: as mg/l malic acid is 0.44.
   Genotype.—‘WA 2’ is homozygous for the ethylene production genes Md-ACS1 and Md-ACO1.
   Keeping quality.—excellent, exceeding 90 days in common storage with little to no loss in firmness and acidity.
Pollination: Any diploid apple of the same bloom season
Use: Primarily for fresh eating
Disease and insect resistance: Considered susceptible to all apple insects and has known susceptibility to apple powdery mildew; susceptibility to other apple diseases is unknown

Table 1 represents fruit indices of the ‘WA 2’, ‘WA 2’ second generation, ‘Gala’, ‘Braeburn’, ‘Cripps Pink’ and ‘Fuji’ varieties at harvest maturity and following 60 days in common storage. The differences between fruit from the ‘WA 2’ mother tree and from the ‘WA 2’ second generation trees are as expected from an apple cultivar grown in different environments.

TABLE 1
Fruit indices at harvest maturity and following 60 days in common storage

| | | | Sugars | | Acidity | | |
|---|---|---|---|---|---|---|---|
| | Firmness | | % Soluble | | mg/l | | |
| | Newtons | | solids | | malic acid | | |
| Cultivar | Fresh | Storage | Fresh | Storage | Fresh | Storage | Year |
| ‘WA 2’ | 77 | 70 | 13.9 | 14.3 | 0.43 | 0.35 | 2008 |
| ‘WA 2’ | 81 | 76 | 13.9 | 14.0 | 0.51 | 0.40 | 2008 |
| 2^{nd} gen. | | | | | | | |
| ‘Gala’ | 72 | 73 | 13.3 | 14.1 | 0.58 | 0.34 | 2008 |
| ‘Braeburn’ | 86 | 82 | 14.5 | 15.4 | 0.87 | 0.51 | 2008 |
| ‘Cripps Pink’ | 84 | 82 | 15.6 | 16.0 | 0.94 | 0.66 | 2008 |
| ‘Fuji’ | 72 | 64 | 14.5 | 15.7 | 0.56 | 0.46 | 2008 |

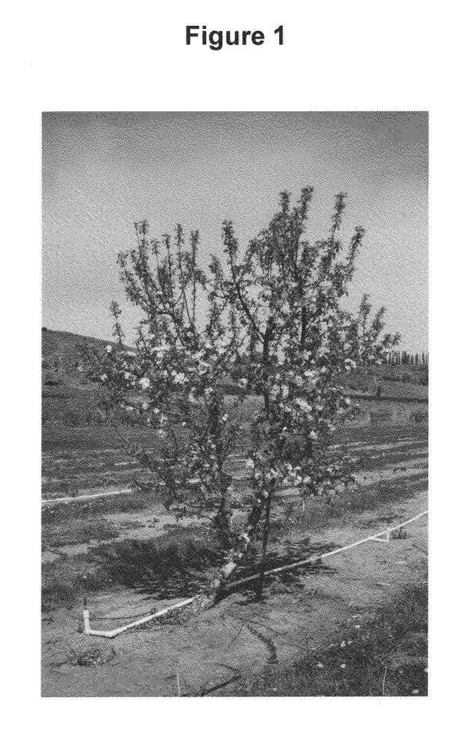

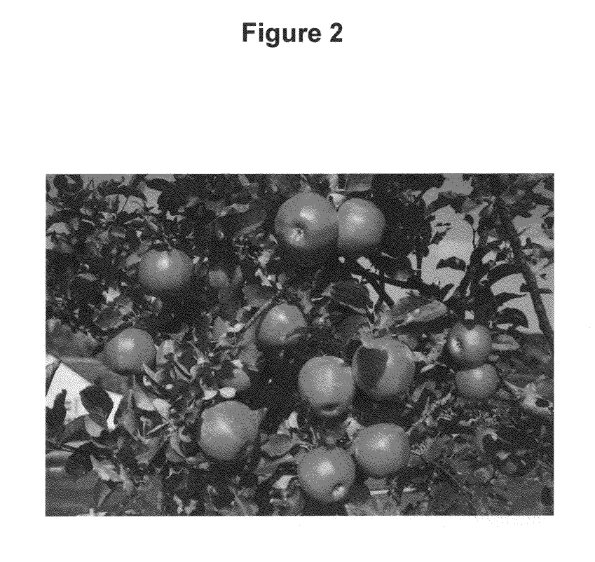

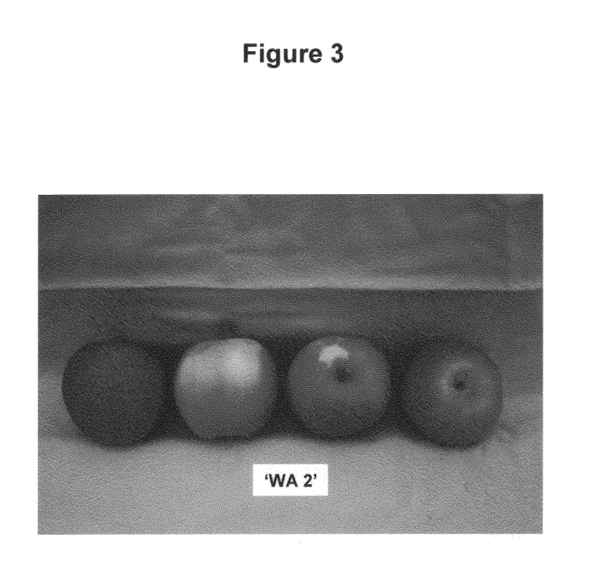

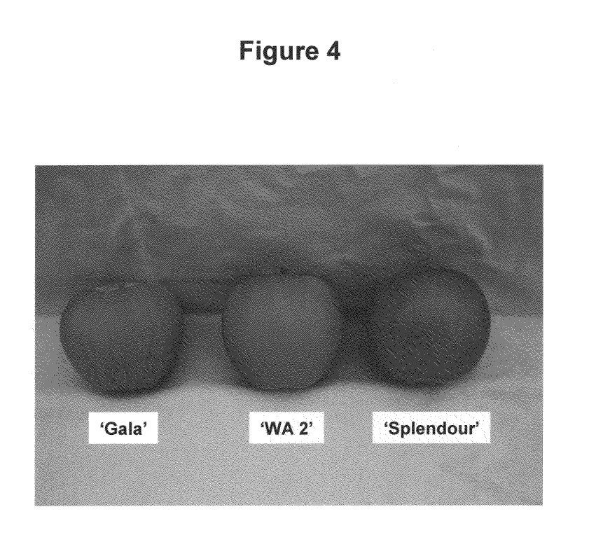

The attached figures show the ‘WA 2’ tree and various aspects of its fruit. FIG. 1 is a photo of the eight year old mother tree of ‘WA 2’. FIG. 2 is a close up of the apples at harvest maturity on the mother tree. FIG. 3 shows representative views of the exposed and backsides of the ‘WA 2’ fruit, in addition to the stem and calyx ends of the fruit. FIG. 4 shows the ‘WA 2’ fruit as compared to the fruit of its two parental varieties at harvest maturity. A typical ‘Tenroy Gala’ apple, suspected of being the male parent cultivar, and a typical ‘Splendour’ apple, are show next to a ‘WA 2’ apple.

What is claimed:

1. A new and distinct apple tree variety named ‘WA 2’, as herein shown and described.
